(19) United States
(12) Patent Application Publication
(10) Pub. No.: US 2025/0052730 A1
(43) Pub. Date: Feb. 13, 2025

(54) Methods for Detecting a Protein in a Sample in a Fluidic Device Using Mass Spectrometry

(71) Applicant: AMGEN, INC., Thousand Oaks, CA (US)

(72) Inventor: Iain D.G. Campuzano, Thousand Oaks, CA (US)

(21) Appl. No.: 18/720,034

(22) PCT Filed: Dec. 22, 2022
(86) PCT No.: PCT/US2022/053745

§ 371 Date: Jun. 14, 2024

Related U.S. Application Data
(60) Provisional application No. 63/293,502, filed on Dec. 23, 2021.
(60) Provisional application No. 63/432,522, filed on Dec. 14, 2022.

(51) Int. Cl. G01N 30/72; B01D 15/32; B01D 15/36; G01N 30/02; G01N 30/88; G01N 33/68; H01J 49/00; H01J 49/16

(52) U.S. Cl. CPC G01N 30/7266; B01D 15/327; B01D 15/362; G01N 30/88; G01N 33/6851; G01N 33/6854; H01J 49/004; H01J 49/164; G01N 2030/027; G01N 2030/8831

(57) ABSTRACT

The disclosure provides for methods of detecting and analyzing proteins using mass spectrometry analysis, such as matrix assisted laser desorption ionization mass spectrometry (MALDI-MIS) on protein directly within a fluidic device, or reversed phase liquid chromatographic mass spectrometry (rpLC-MS) on protein outside of the fluidic device.

The application claims priority to U.S. Provisional Application No. 63/293,502, filed Dec. 23, 2021 and U.S. Provisional Application No. 63/432,522, filed Dec. 14, 2022, both of which are incorporated herein by reference in their entirety.

### FIELD

The disclosure provides for methods of detecting and analyzing proteins using mass spectrometry analysis, such as matrix assisted laser defcasorption ionization mass spectrometry (MALDI-MS) on protein directly within a fluidic device, or reversed phase liquid chromatographic mass spectrometry (rpLC-MS) on protein outside of the fluidic device.

### BACKGROUND

MALDI-MS and rpLC-MS is a widely used technique for determining the molecular weight (Mw) of biomolecules, with exceptional capabilities for rapid Mw determination of small molecules, peptides and proteins. Mass spectrometry imaging, such as MALDI-MS imaging, is used on thin tissue slices that have been immobilized on a MALDI-MS target plate. A number of analytical techniques are based on forces exerted by a light beam (known as optical manipulations), which enable interactive biology at the cellular level, thus opening new opportunities in drug discovery. Optical manipulation which permits highly selective and dynamic processes in micro- and nanoscopic systems—has proven to be a versatile and integrated technology throughout many scientific areas. This technology is based on light-induced electrokinetics that gives rise to designated forces on both solid and fluidic structures (1). For example, the commercially available fluidic devices, such as the integrated technology of the Berkeley Lights (BLI) Beacon® Optofluidic System (Emeryville, CA) have the flexibility and capability for a broad array of applications applicable to commercial large molecule drug development, including antibody discovery, clonal selection, gene editing, linking phenotype to genotype, and cell line development. RPLC-MS is a widely used technique for also determining the Mw's of pharmaceutical and bio pharmaceutically relevant molecules (12)

### SUMMARY

There is a need to develop “on-microchip” assays predictive of molecule performance in shake flasks and bioreactors. Disclosed herein are methods using mass spectrometry, such as MALDI-MS on protein in a fluidic device. In addition, the disclosed methods allow for the protein-producing cells to be grown within the fluidic device and mass spectrometry to be carried out directly on media containing the protein such as conditioned media.

In one aspect, disclosed herein are methods of detecting a protein in a fluidic device. In various embodiments, the method comprises subjecting the sample to mass spectrometry while the sample is in a fluidic device. In some embodiments, the protein in the sample is reduced. The methods disclosed herein detect protein level and provide methods for identify changes in the molecular weight (Mw) of the protein or analyze modifications to the protein. For example, when the protein is an antibody, the methods allow for determinations such as antibody heavy and light chain Mw along with the identification of clipping, light chain and heavy chain pairing and mis-pairing (e.g., for multi-specific mAbs) and other post-translational modifications from antibody-producing cells grown on-chip.

In some embodiments, the disclosure provides for methods of detecting protein in samples in a fluidic device comprising: contacting samples in the device with solid supports, each solid support comprising a ligand for the protein, whereby the ligand binds to the protein of the sample, and wherein each solid support comprising the ligand bound to the protein comprises a unique barcode different from other solid supports comprising the ligand bound to the protein; transporting the solid support comprising the ligand bound to the protein from a first location in the fluidic device to a second location, wherein the second location is in the fluidic device or is outside of the fluidic device; and subjecting the sample to mass spectrometry at the second location.

In various embodiments, the methods of detecting a protein further comprise reducing the protein in the sample prior to subjecting the sample to mass spectrometry. For example, the protein may be reduced by contacting the protein in the sample with sinapinic acid and TCEP such as mixing the protein in the sample with TCEP in the presence of sinapinic acid. For example, the protein may be reduced in situ or the protein may be reduced on the mass spectrometry plate, e.g. the MALDI plate, prior to subjecting the sample to mass spectrometry.

The disclosed methods may be carried out with any type of mass spectrometry methods which are electrospray ionization (ESI) enabled. For example, the mass spectrometry for use in the methods described herein include matrix-assisted laser desorption/ionization time-of-flight/time-of-flight (MALDI-TOF/TOF), liquid chromatography-mass spectrometry/mass-spectrometry (rpLC-MS), hydrophobic interaction chromatography-mass spectrometry (HIC-MS) or cation exchange chromatography-mass spectrometry (CEX-MS). In related embodiments, the rpLC-MS comprises electrospray ionization (ESI).

In any of the disclosed methods, the sample is any liquid or formulation comprising a protein. In various embodiments, the sample is a fluid comprising a protein that is to be processed, measured or analyzed for stability and/or structural integrity or other attributes. In some embodiments, the sample comprises or consists of conditioned media or any liquid from which a protein is purified or isolated. In some embodiments, the sample comprises a cell which produces the protein.

In any of the disclosed methods, the protein comprises or consists of an antibody, antibody protein product, bispecific T-cell engager (BiTE®) molecule, antibody fragment, antibody fusion peptide or antigen-binding fragment thereof, peptide, growth factor, or cytokine. In related embodiments, the antibody is a polyclonal or monoclonal antibody. As used herein, the term “antibody protein product” refers to any one of several antibody alternatives which in various instances is based on the architecture of an antibody but is not found in nature. In some aspects, the antibody protein product has a molecular-weight within the range of at least about 12 kDa-1 MDa, for example at least about 12 kDa-750 KDa, at least about 12 kDa-250 kDa, or at least about 12 kDa-150 kDa. In certain aspects, the antibody protein product has a valency (n) range from monomeric (n=1), to dimeric (n=2), to trimeric (n=3), to tetrameric (n=4), if not higher order valency. Antibody protein products in some aspects are those based on the full antibody structure and/or those that mimic antibody fragments which retain full antigen-binding capacity, e.g., scFvs, Fabs and VHH/VH (discussed below). The smallest antigen binding antibody fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats comprising scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody comprises a biologically active peptide grafted onto an Fc domain. Peptibodies are well-described in the art. See, e.g., Shimamoto et al., mAbs 4(5): 586-591 (2012). Other antibody protein products include a single chain antibody (SCA); a diabody; a triabody; a tetrabody; bispecific or trispecific antibodies, and the like. Bispecific antibodies can be divided into five major classes: BslgG, appended IgG, BsAb fragments, bispecific fusion proteins and BsAb conjugates. See, e.g., Spiess et al., Molecular Immunology 67(2) Part A: 97-106 (2015). In exemplary aspects, the antibody protein product comprises or consists of a bispecific T cell engager (BITE®) molecule, which is an artificial bispecific monoclonal antibody. BITE® molecules are fusion proteins comprising two scFvs of different antibodies. One binds to CD3 and the other binds to a target antigen. BiTE® molecules are known in the art. See, e.g., Huehls et al., Immuno Cell Biol 93 (3): 290-296 (2015); Rossi et al., MAbs 6(2): 381-91 (2014); Ross et al., PLOS One 12(8): e0183390.

In various embodiments, the protein is partially purified prior to being subjected to mass spectrometry. The term “purify” refer to the isolation or separation of a protein from the components of mixtures comprising the protein, such mixtures include crude materials, cell lysates, conditioned media, or other cell culture material comprising the protein. The term “partially purify” refers to removing some but not all of the components of the mixture comprising the protein. The components removed from the mixtures include cellular debris, protein aggregates, fats and/or protease.

In some embodiments, the disclosed method further comprise partially purifying the protein. The purification step can be carried out with any method known in the art. For example, the partial purification may comprise contacting the sample with a solid support (e.g., bead) comprising a ligand for the protein, prior to subjecting the sample to mass spectrometry. As used herein, the “ligand” for the protein refers an agent that binds to the protein such as an antibody that binds the protein or a binding partner. For example, if the protein comprises or consists of a cytokine, an example of a ligand for purposes herein includes the corresponding cytokine receptor (or a binding fragment thereof). For example, if the protein comprises or consists of an antibody, examples of ligand for purposes herein include an antigen for that antibody, an anti-idiotype antibody, anti-Fc antibody, protein A, or protein G. In exemplary embodiments, the solid support (e.g., bead) comprises anti-FC protein, protein A or protein G, or the solid support (e.g., bead) comprises protein A or protein G.

In some embodiments, the solid support comprising the ligand bound to the protein comprises a unique barcode, wherein the partial purification further comprises other solid supports comprising ligand bound to other protein and comprising other barcodes that are different from the unique barcode.

In some embodiments, the disclosed methods the subjecting the sample to mass spectrometry comprises subjecting a batch comprising the solid support, wherein the solid support comprising the ligand bound to the protein comprises a unique barcode and wherein the solid support further comprises at least some of the other solid supports comprising ligand bound to other protein and comprising other barcodes. The term “batch” refers to multiple solid supports, which comprise sample from multiple wells or containers.

For example, the partial purification may comprise contacting the sample with a solid support (e.g., bead) comprising a ligand for the protein, prior to subjecting the sample to mass spectrometry. In some embodiments, the partial purification further comprises transporting the solid support (e.g., bead) comprising the ligand bound to the protein from a first location in the fluidic device to a second location in the fluidic device prior to subjecting the sample to mass spectrometry. The second location may be a second fluidic chip or a second fluidic plate or a second region, flow path, channel, chamber or pen in the fluidic device. In addition, the second location is outside of the fluidic device, for example a well in a multi-well plate such as a 96- or 384-well plate.

In any of the disclosed methods, the method can be carried out with any fluidic device or fluidic apparatus known in the art. A fluidic device (or fluidic apparatus) is a device that includes one or more discrete circuits configured to hold a fluid, each circuit comprised of fluidically interconnected circuit elements. The circuit element including but not limited to region(s), flow path(s), channel(s), chamber(s), and/or pen(s), and at least one port configured to allow the fluid to flow into and/or out of the fluidic device. The fluidic circuit may be configured to have a first end fluidically connected with a first port (e.g., an inlet) in the microfluidic device and a second end fluidically connected with a second port (e.g., an outlet) in the fluidic device or connected to a second fluidic device or a second region, flow path, channel, chamber or pen in the fluidic device. The fluidic device may be a microfluidic device, through other scales such as nano-scale may also be suitable. For example, the fluidic device may be a microfluidic chip, microfluidic channel, microfluidic cell, nanofluidic chip, nanofluidic channel, nanofluidic cell or sequestration pen. The discrete circuit or circuits of the fluidic device may comprise silicon, such as a silicon surface configured for the sample to be disposed thereon. For example, the discrete circuit or circuits may comprise a flow region defined by at least one silicon surface. By way of example, the discrete circuit or circuits may comprise a microfluidic channel or nanofluidic channel defined by at least one silicon surface. In some embodiments, the microfluidic channel or nanofluidic channel is etched in silicon. It will be understood that mass spectrometry may be performed on a silicon surface (See, e.g, Lewis et al., “Desorption/ionization on silicon (DIOS) mass spectrometry: background and applications.” International Journal of Mass Spectrometry 226 (2003) 107-116). As such, in the method of some embodiments, the sample is disposed on a silicon surface of the fluidic device.

For a microfluidic device the circuit will include a flow region, which may include a microfluidic channel, and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 μL. In certain embodiments, the microfluidic circuit holds about 1-2, 1-3, 1-4, 1-5, 2-5, 2-8, 2-10, 2-12, 2-15, 2-20, 5-20, 5-30, 5-40, 5-50, 10-50, 10-75, 10-100, 20-100, 20-150, 20-200, 50-200, 50-250, or 50-300 μL. The microfluidic circuit may be configured to have a first end fluidically connected with a first port (e.g., an inlet) in the microfluidic device and a second end fluidically connected with a second port (e.g., an outlet) in the microfluidic device.

As used herein, a “nanofluidic device” or “nanofluidic apparatus” is a type of fluidic device having a fluidic circuit that contains at least one circuit element configured to hold a volume of fluid of less than about 1 μL, e.g., less than about 750, 500, 250, 200, 150, 100, 75, 50, 25, 20, 15, 10, 9, 8, 7, 6, 5, 4, 3, 2, 1 nL or less. A nanofluidic device may comprise a plurality of circuit elements (e.g., at least 2, 3, 4, 5, 6, 7, 8, 9, 10, 15, 20, 25, 50, 75, 100, 150, 200, 250, 300, 400, 500, 600, 700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 pL to 1 nL, 100 pL to 2 nL, 100 pL to 5 nL, 250 pL to 2 nL, 250 pL to 5 nL, 250 pL to 10 nL, 500 pL to 5 nL, 500 pL to 10 nL, 500 pL to 15 nL, 750 pL to 10 nL, 750 pL to 15 nL, 750 pL to 20 nL, 1 to 10 nL, 1 to 15 nL, 1 to 20 nL, 1 to 25 nL, or 1 to 50 nL. In other embodiments, one or more (e.g., all) of the at least one circuit elements is configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A “fluidic channel” or “flow channel” as used herein refers to a flow region of a fluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is in the range of from about 50,000 microns to about 500,000 microns, including any range there between. In some embodiments, the horizontal dimension is in the range of from about 100 microns to about 1000 microns (e.g., about 150 to about 500 microns) and the vertical dimension is in the range of from about 25 microns to about 200 microns, e.g., from about 40 to about 150 microns. It is noted that a flow channel may have a variety of different spatial configurations in a fluidic device, and thus is not restricted to a perfectly linear element. For example, a flow channel may include one or more sections having any of the following configurations: curve, bend, spiral, incline, decline, fork (e.g., multiple different flow paths), and any combination thereof. In addition, a flow channel may have different cross-sectional areas along its path, widening and constricting to provide a desired fluid flow therein

The disclosure also provides for a MALDI mass spectrometry target plate comprising a fluidic device mounted to the surface of the mass spectrometry MALDI target plate. In some embodiments, the fluidic device is open, thereby configuring an interior of the fluidic device to be directly engaged by a mass spectrometer. For example, the mass spectrometry for use in the methods described herein include matrix-assisted laser desorption/ionization time-of-flight/time-of-flight (MALDI-TOF/TOF) or liquid chromatography-mass spectrometry/mass-spectrometry (LC-MS/MS). In related embodiments, the LC-MS/MS comprises electrospray ionization.

In addition, the disclosure provides for a system for detecting protein in a sample wherein the system comprises any of the disclosed mass spectrometry plates disclosed herein. In some embodiments, the system further comprises a control or a protein standard. In addition, in various embodiments, the system further comprises a solid support (e.g., bead) comprising a ligand, such as anti-Fc protein, protein A or protein G.

The disclosure also provides for a kit comprising a mass spectrometry plate disclosed herein. In various embodiments, the kit further comprises a solid support (e.g., bead) comprising a ligand, such as anti-Fc protein, protein A or protein G.

### BRIEF DESCRIPTION OF THE DRAWINGS

FIG. 1 is a mass spectrum from MALDI-MS analysis of protein spotted on the MALDI plate. The top panel is a mass spectrum for protein at 1 mg per mL, the middle panel is a mass spectrum for protein at 0.5 mg/mL, and the bottom panel is a mass spectrum for protein at 0.125 mg/mL

FIG. 2 is a mass spectrum from MALDI-MS analysis of conditioned media containing expressed mAb. The spectrum was dominated by the light chain while the signal corresponding to the intact antibody (as indicated by the arrow) was suppressed.

FIGS. 3A and 3B are a RP-HPLC chromatogram from separation of Day 11 conditioned media containing expressed antibody and a reducing SDS-PAGE identifying the eluted RP-HPLC fractions. FIG. 3A, shows detection of three major peaks in the conditioned media containing expressed antibody. FIG. 3B shows reducing SDS-PAGE identification of the RP-HPLC fractions from peaks 3a & 3b as corresponding in size with the purified mAb control in the last lane.

FIGS. 4A-4B show rpLC-MS ESI spectra from the analysis of peak 1 and 2 RP-HPLC eluates by LC-ESI-MS. FIG. 4A identifies peak 1 as a mixture of cysteinylated and glutathionylated light chain. FIG. 4B identifies peak 2 as the light chain dimer and the dimer with cysteine or glutathione adduct.

FIG. 5 shows a RP-HPLC chromatogram and an ESI spectra from conditioned media analysis following affinity purification with a Protein A/G spin column. Top panel shows recovery of expressed mAb from conditioned media using a Protein A/G spin column with the light chain derivatives eluting in the unbound fraction. Bottom panel is RP-HPLC which confirms mAb presence in the bound fraction after elution with 1% acetic acid.

FIG. 6 provides RP-HPLC chromatograms showing recovery of 80% of the mAb from the conditioned media by binding to Pierce protein A/G magnetic beads. Upper panel shows the abundance of the mAb in the conditioned media prior to the addition of the magnetic beads (peak 3). Lower panel, shows reduction of mAb abundance in the conditioned media (depletion of peak 3) after addition of the magnetic beads, demonstrating that most of the mAb binds to the beads.

FIG. 7 is a MALDI-MS spectrum from direct analysis of protein A/G magnetic beads with bound mAb displaying the Mw of the intact mAb.

FIG. 8 is a MALDI-MS spectrum from on-plate reduction of mAb bound on the Pierce protein A/G magnetic beads.

FIG. 9 is RP-HPLC chromatograms showing recovery of the mAb from the conditioned media by binding to Promega Protein A beads. Peak 1 is the mAb light chain. Peak 2 is the mAb light chain dimer. Peak 3 is the mAb of interest. Upper panel shows the abundance of the mAb in the conditioned media prior to the addition of the Protein A beads. Lower panel shows reduction of mAb abundance in the conditioned media (dramatic depletion of peak 3) after addition of Protein A beads, demonstrating that most of the mAb binds to the beads.

FIG. 10 is a MALDI-MS spectrum from direct analysis of Promega Protein A magnetic beads with bound mAb showing the molecular Mwof the intact mAb.

FIG. 11 is a MALDI-MS spectrum of mAb after acid elution from Protein A magnetic beads prior to analysis.

FIG. 12 shows overlaid spectra obtained from direct analysis of bead bound mAb and a 3-fold increase in signal with the inclusion of an acid elution step prior to analysis.

FIG. 13 shows MALDI-MS of mAb after reduction directly on glass cover of the MALDI-MS plate.

FIG. 14 shows a BLI chip surface mount to a MALDI-MS target plate.

FIG. 15 shows a BLI chip mount into a rear pocket (“picture-frame”) in the MALDI-MS target plate.

### DETAILED DESCRIPTION

Mass spectrometry imaging, such as MALDI-MS imaging, is commonly used on thin tissue slices that have been immobilized on a MALDI-MS target plate. Disclosed herein are methods of detecting a protein in a sample by subjecting the sample to mass spectrometry while the sample is in a fluidic device. In the present methods, systems and devices, the MALDI-MS target plate can be modified to house/mount the BLI chip. For example, the MALDI laser is typically 10-20 μm in diameter (special focus) and the microfluidic pens are approximately 60 μm in width, therefore the laser dimensions “fit” inside the BLI pens.

If one can analyze/measure the Mw of molecules such as mAbs and/or multi-specifics by MALDI-MS directly from the BLI pen, one can determine when the molecule is the expected molecule, based on the measured Mw. Levels of chain pairing and mispairing and post-translational modifications (PTMs) can potentially be identified. Mass spectrometry accuracy and sensitivity can be improved by reducing the mAb on the target plate, or the antibody may be reduced in situ, e.g. reduced while on a solid substrate or the antibody may be reduced directly on the mass spectrometry plate.

In some embodiments, the mass spectrometry plate is configured to accommodate the fluidic device. For example, the fluidic device, fluidic chip, or fluidic chamber or channel or the sequestration pen may be mounted or machined onto the mass spectrometry plate. For example, the mass spectrometry plate may be configured for mounting a fluidic device thereon. In some embodiments, the fluidic device is mounted to the surface of the MS plate or MS matrix. In other embodiments, the fluidic device is mounted into a rear pocket of the mass spectrometry plate. In these embodiments, the fluidic device may be open or may comprise a removable cover to insert MS matrix and/or permit access by the mass spectrometer. The removable cover may comprise a glass, crystal, or polymer cover slip.

In other embodiments, the liquid MS matrix is flowed directly into the fluidic device. For example, when the MS is MALDI-MS and the fluidic device is a chip or sequestration pen, the liquid MALDI matrix is directly flowed into the chip or pens using a microfluidic system, such as the BLI Beacon® Microfluidics system. For fluidic chips that comprise a removable cover, the cover may be removed before or after the MS matrix has been flowed therein.

The fluidic device allows for growing and expanding a single cell within a chamber or sequestration pen, which in turn allow for clonal selection of the cell producing the protein to be detected. The clonal selection allows for selection of the clones for large-scale protein production and purification during drug discovery and biologic drug manufacturing, e.g. antibody production. The mass spectrometry analysis allows for analysis of modifications to the proteins, such as a post-translational modification that results in a change in the Mw of the protein being analyzed e.g. glycosylation, cysteinylation or glutathionylation, oxidation, deamination, glycation, phosphorylation, sulphation or ubiquitination. In addition, the mass spectrometry analysis allows for determination of mispaired species and fragmented proteins. The disclosed methods also allow for continual analysis of the cells as they are expanding and the assays can be repeated on the same growing cell.

A colony of biological cells is “clonal” if all of the living cells in the colony that are capable of reproducing are daughter cells derived from a single progenitor cell. In certain embodiments, all the daughter cells in a clonal colony are derived from the single parent cell by no more than 10 divisions. In other embodiments, all the daughter cells in a clonal colony are derived from the single progenitor cell by no more than 14 divisions. In other embodiments, all the daughter cells in a clonal colony are derived from the single progenitor cell by no more than 17 divisions. In other embodiments, all the daughter cells in a clonal colony are derived from the single progenitor cell by no more than 20 divisions. The term “clonal cells” refers to cells of the same clonal colony.

As used herein, a “colony” of biological cells refers to 2 or more cells (e.g. about 2 to about 20, about 4 to about 40, about 6 to about 60, about 8 to about 80, about 10 to about 100, about 20 about 200, about 40 about 400, about 60 about 600, about 80 about 800, about 100 about 1000, or greater than 1000 cells).

As used herein, the term “maintaining (a) cell(s)” refers to providing an environment comprising both fluidic and gaseous components and, optionally a surface, that provides the conditions necessary to keep the cells viable and/or expanding.

As used herein, the term “expanding” when referring to cells, refers to increasing in cell number.

### Mass Spectrometry

In exemplary aspects, the method comprises using mass spectrometry to detect a protein in a sample. Disclosed herein are methods of subjecting a sample to mass spectrometry when the sample is in a fluidic device. Mass spectrometry is used to measure the mass/charge of ions. The methods herein may comprise ESI enabled mass spectrometry.

Matrix assisted laser desorption ionization mass spectrometry (MALDI-MS) is a simple, effective, and widely used technique for determining the Mw of biomolecules, with exceptional capabilities for rapid Mw determination of small molecules (2), peptides and proteins (3). First described by Karas and Hillenkamp in 1985 where MALDI was used to analyze a selection of amino acids and dipeptides (4). In various embodiments, the method comprises MALDI-MS.

MALDI-MS imaging has the ability to analyze many different tissue specimens/samples. In brief, a thin tissue slice is fixed on to a MALDI-MS target plates, coated with matrix and ablated with a laser (within the mass spectrometer). Detection is typically using a time-of-flight (ToF) analyxer, however MALDI has been combined with both ion cyclotron resonance (ICR) (5) and Orbitrap (6) MS systems. A very wide range of analytes, from proteins, peptides, protein modification, small molecules, drugs and their metabolites as well as pharmaceutical components, endogenous cell metabolites, lipids, and other analytes are made accessible by MALDI-MS imaging of thin tissue slices (7). This method is label-free and allows multiplex analysis of hundreds to thousands of molecules in the very same tissue section simultaneously. In various embodiments, the mass spectrometry comprises or consists of MALDI-MS. In various embodiments, the mass spectrometry comprises or consists of matrix-assisted laser desorption/ionization time-of-flight/time-of-flight (MALDI-TOF/TOF). The MALDI TOF/TOF is an efficient method for detecting fragmented peptides. In various embodiments, the mass spectrometry comprises or consists of liquid chromatography-mass spectrometry/mass-spectrometry (LC-MS/MS), also referred to as liquid chromatography-tandem mass spectrometry. In the present disclosure, the two terms MALDI-TOF/TOF and MALDI-LIFT-TOF/TOF are used interchangeably.

### Sample

The sample may comprise any type of protein that may be measured, processed or analyzed for stability and/or structural integrity. In some embodiments, the sample comprises or consists of conditioned media or any liquid from which the protein may be purified or isolated. In various embodiments, the protein sample so subjected to the methods disclosed herein, comprises or consists of a large peptide, antibody, antibody fragment, antibody fusion peptide or antigen-binding fragments thereof. In related embodiments, the antibody is a polyclonal or monoclonal antibody.

In various embodiments, the protein within the sample is reduced. The protein is reduced to improve sensitivity. The protein is reduced using any method known in the art. For example, the protein is reduced with redox agents such as dithiothreitol (DTT), ß-mercaptoethanol and TCEP (Tris (2-carboxyethyl) phosphine) in sinapinic acid. In addition, the protein is reduced by mixing the protein in the sample with TCEP in sinapinic acid and TCEP or contacting the protein in the sample with acetic acid. For example, the protein is released from the solid support (e.g. beads) with acetic acid followed by the addition of TCEP and sinapinic acid. The protein may be reduced prior to being inserted on the mass spectrometry plate or the protein may be reduced after being inserted on the mass spectrometry plate.

### Fluidic Devices

Fluidic devices refer to an apparatus that use small amounts of fluid to carry out various types of analysis. The fluidic device comprises one or more discrete circuits configured to hold a fluid, each circuit comprised of fluidically interconnected circuit elements. The circuit element including but not limited to region(s), flow path(s), channel(s), chamber(s), and/or pen(s), and at least one port configured to allow the fluid to flow into and/or out of the fluidic device. These devices use chips, cells, channel, or sequestrian pens that contain the fluid for analysis.

Microfluidic devices generally have one or more channels with at least one dimension less than 1 mm. Common fluids used in microfluidic devices include whole blood samples, bacterial cell suspensions, protein or antibody solutions and various buffers. Microfluidic devices can be used to obtain a variety of measurements including molecular diffusion coefficients, fluid viscosity, pH, chemical binding coefficients and enzyme reaction kinetics. Other applications for microfluidic devices include capillary electrophoresis, isoelectric focusing, immunoassays, flow cytometry, sample injection of proteins for analysis via mass spectrometry, PCR amplification, DNA analysis, cell manipulation, cell separation, cell patterning and chemical gradient formation. Many of these applications have utility for clinical diagnostics.

The advantages for using microfluidic devices include that the volume of fluids within these channels is very small, usually several nanoliters, and the amounts of reagents and analytes used is quite small. Moreover, when analyzing protein-producing cells, a relatively small number of cells (or even single cells) can produce a sufficient quantity and concentration of protein for analysis, reducing or avoiding incubation times for colony expansion. The fabrications techniques used to construct microfluidic devices, discussed in more depth later, are relatively inexpensive and are very amenable both to highly elaborate, multiplexed devices and also to mass production. Microfluidic technologies enable the fabrication of highly integrated devices for performing several different functions on the same substrate chip.

Any fluidic device can be used (or modified to be used) in the disclosed methods, including commercially available devices. The fluidic device may be configured for use in an optofluidic system, which can use light to manipulate matter in the fluidic device such as cells. Described herein as an exemplary microfluidic device is a chip comprising the Berkley Lights (BLI) pen. For example, the BLI pen may be analyzed in The Beacon® Optofluidic System, the Lightning™ Optofluidic System or the Culture Station System (BLI. Emeryville, CA). Other exemplary optofluidic systems are the Cyto-Mine® System (Sphere Fluidics, Great Abington, Cambridge, UK).

In any of the disclosed methods, a single cell or a low number of cells (˜1-10 cells) is seeded in the fluidic device, and these cells express the protein that is to be detected. The proteins expressed by the cells within the fluidic device are sampled directly by MS while in the fluidic device using any of the disclosed methods. In this scenario, the expressed protein in the fluidic device needs to be to be fixed and/or coated with the MALDI matrix prior to MS analysis. The fluidic device, such as the BLI chip, is then loaded on to/in to the existing MALDI-MS target plate and loaded in the MS instrument for subsequent analysis. Depending on how the overexpressed protein in the fluidic device is treated (with or without reductant in the matrix), the protein can be analyzed under either reducing conditions or non-reducing conditions. For example, when the protein to be analyzed is a monoclonal antibody, the monoclonal antibody can be analyzed under either reducing condition (light and heavy chain) or non-reducing (intact) conditions.

In various embodiments, the MS carried out in the disclosed method is MALDI-MS. The special focus of the MALDI laser makes this type of MS very suitable for efficiently carrying out the disclosed methods. Another feature to consider for the disclosed methods is the dimensions of the fluidic device, e.g. the sequestration pen. Modern lasers within MALDI MS instrument are typically solid-state nitrogen-based Nd-YAG/YLF (neodymium-doped yttrium aluminum garnet/yttrium lithium fluoride) lasers have been documented to achieve a laser spot size of 1.1 μm to 8.4 μm (8, 9). The dimensions of the available microfluidic chips include the following: 130×370 μm; 50×370 μm; 40×200 μm; 40×160 μm (these sizes are commercially available from BLI, Emeryville, CA). Therefore, based on the available sequestration pen dimensions, modern MALDI-MS lasers will easily be able to “fit” inside the current pens and be used to ablate/analyze matrix fixed protein. Additionally, the current repetition rates of modern lasers are up to 5 kHz (10), allowing for the complete analysis of the most populated available fluidic chip (40×160 μm) in a matter of hours.

### Partial Protein Purification and Pooling of Samples

In some embodiments, the disclosed method comprises partially purifying the protein in the sample prior to subjecting the sample to MS. The partial purification can be carried out with any method known in the art. For example, the partial purification may comprise contacting the sample with a solid support, such as a bead or resin, comprising a ligand for the protein, prior to subjecting the sample to mass spectrometry. The ligand for the protein may be an agent that binds to the protein such as an antibody that binds the protein or a binding partner. In exemplary embodiments, the protein is a monoclonal antibody and the solid support is a bead comprising anti-Fc protein, protein A or protein G, and the partial purification comprises transporting the bead comprising the ligand bound to the antibody from a first location in the fluidic device to a second location in the fluidic device prior to subjecting the sample to MS.

Moving the solid supports (e.g., beads) out of the fluidic device and on to a separate MS target plate or into a 96 well and/or 384 well plate for (reversed-phase liquid chromatographic mass spectrometry) rpLC-MS analyses (11, 12). Moving the solid support (e.g., bead) captured proteins out of the fluidic device allows for “pooling” and therefore greater MS sensitivity. Mobile phases for rpLC-MS are acidic in nature, therefore this is compatible with eluting the captured protein from the solid support (e.g., bead).

In addition, in any of the disclosed methods, the MS may be tandem-MS (both MALDI-MS/MS and rpLC-MS/MS) which is carried out to obtain or confirm protein sequence information and/or to identify or quantify post translational modification identification and quantification. “Top-down MS/MS” refers to MS/MS performed on a non-reduced sample. “Middle-down MS/MS” refers to MS/MS performed on a reduced material. “Bottom-up MS/MS refers to MS/MS performed on a reduced and proteolytically digested material.

Suitable solid supports for purifying or partially purifying the protein include for example, beads or resins. When the solid support is a bead, the beads may be of a size that a single bead may comprise a quantity of protein for MS. For example, as shown in the Examples herein, beads having a mean diameter of at least about 1 μm may comprise sufficient quantities of protein for analysis by MS. For example, the solid support may comprise or consist of beads having mean diameters of at least about 1 μm, 2 μm, 3 μm, 4 μm, 5 μm, 10 μm, 20 μm, or 40 μm, including ranges between any two of the listed values, for example 1-5 μm, 1-10 μm, 1-20 μm, 1-40 μm, 5-10 μm, 5-20 μm, 5-40 μm, 10-20 μm, or 10-40 μm. For any of the solid supports, a ligand for the protein may be immobilized on the solid support, for example by a covalent linkage. In some methods, the beads may be magnetic beads. When the solid support is a resin, the resin may be a polymeric resin such as cellulose, polystyrene, agarose, and polyacrylamide or agarose.

In some methods, the MS may be performed on a batch of solid supports each comprising different proteins. For example, single beads comprising ligand may each be bound to protein in a discrete location of a fluidic device such as a pen, so that each single bead comprises protein produced by a different clone or colony of clonal cells. The single beads may then be pooled at a second location and analyzed at that second location (which may be in the fluidic device or outside of the fluidic device). In such methods, the bead or beads associated with each discrete location (e.g., pen) may be barcoded so that protein originating from a clone in that location may be identified from the pool. For example, the protein in each location may comprise a unique peptide or PTM barcode, or each bead or the ligand thereon may comprise a unique barcode. Examples of suitable barcodes include peptides or nucleic acids having unique sequences or molecular weights, pigments or combinations of pigments, glycans and/or carbohydrates or combinations thereof or fluorophores or combinations of fluorophores. In some embodiments, the beads are sized so that each discrete location (e.g., pen) on the fluidic device comprising protein of a different clone can only accommodate a single bead. For example, each bead may have a diameter at least half the diameter of the discrete location so that no more than one bead may be disposed in the discrete location at a time. Each bead may comprise a unique barcode. In some embodiments, the beads are barcoded in the location comprising the protein. For example, each location (e.g., pen) may comprise a unique peptide or nucleic acid barcode (produced by the cell or clonal cells at that location), which becomes immobilized on the bead in situ.

Some methods comprise samples in the device with solid supports, each solid support comprising a ligand for the protein. The ligand may bind to the protein of the sample, and each solid support comprising the ligand bound to the protein may comprise a unique barcode different from other solid supports comprising the ligand bound to the protein. The solid support comprising the ligand bound to the protein may be moved from a first location in the fluidic device to a second location. MS (either MALDI-MS or LC-MS) may be performed on the protein at the second location. The second location may be in the fluidic device, for example, a region of the fluidic device that is open, or that comprises a removable cover, so that the protein in that region may be analyzed by MS (either MALDI-MS or LC-MS). Alternatively, the second location may be outside of the fluidic device, for example a well in a multi-well plate such as a 96- or 384-well plate. The solid supports may be analyzed in batch by MS at the second location. The solid supports may be used to determine the clonal origin of the protein of each bead.

### EXAMPLES

### General Methods

Protein A/G magnetic beads (catalog 88802) were purchased from Thermo Fisher. Magne® Protein A beads (catalog G8781) were obtained from Promega.

### Partial Purification of mAb Using Magnetic Beads.

Magnetic bead (20 μL) slurry was transferred to a 1.5 mL microfuge tube, and subsequently washed with 500 μL of PBS. Following mixing using a vortex mixer, the tube was placed in a magnetic stand where the beads were settled as a “thin strip”. The liquid was removed and 100 μL of conditioned media was added to the beads, followed by gently rocked at room temperature for 30 to 60 minutes. After the beads settled, the liquid portion (the unbound sample) was removed and analyzed using RP-HPLC. The beads were then washed with 500 μL of PBS. After the beads settled, the liquid portion (the wash) was carefully removed. To the remaining beads, which now contain bound mAb, was added 20 μL of PBS, were mixed to generate a slurry. The slurry (1 μL) was spotted onto a MALDI plate, followed by 2 μL of matrix (Sinapinic acid at 10 mg/mL in 0.1% TFA 50% acetonitrile) and 1 μL of 5% acetic acid. The spot was dried at room temperature prior to MALDI-MS.

### On-Plate Reduction of a mAb

The beads with bound mAb were placed onto the MALDI plate, and the beads were contacted with Sinapinic acid (2 μL) (as described above) and 20 mM of TCEP. The sample and thus the reduction was allowed to dry at room temperature. The dried spot was then subjected to MALDI-MS.

Alternatively, the mAb was diluted in 1% acetic acid (10 μL) and 0.1 M TCEP. The sample was incubated at 37° C. for 20 minutes then placed (1 μL) onto the MALDI plate and allowed to dry at room temperature. Sinapinic acid (1 μL) was layered onto the spot, air dried and mass measured by MALDI-MS.

### MALDI-MS Instrumentation

MALDI-MS was performed using a Bruker UltrafleXtreme TOF/TOF MALDI mass spectrometer. The instrument was equipped with a 2000 Hz Smartbeam II (Nd: YAG) laser and was operated in the linear positive mode and calibrated using BSA as external standard. The acceleration voltage used was set at 25 kV. The laser power was optimized and usually 5000 to 8000 laser shots were collected for each sample.

All LC-MS data was acquired on an Agilent 6224 TOF LC/MS system with a 1290 Infinity LC system. Chromatographic separation was achieved using a Zorbax SB300-C8 3.5 μm 2.1×50 mm column operated at a temperature of 70° C. The solvents used were as follows: mobile phase A was water containing 0.1% v/v TFA. Mobile phase B was 90% n-propanol containing 0.1% v/v TFA. Initial gradients conditions were 20% mobile phase B from 0.0 to 1.0 minutes; 1.0 to 9.0 minutes, 20-70% mobile phase B; 9.0-10.0 minutes, 70-100% mobile phase B, where it remains at 100% for 1 further minute. The flow rate was 0.4 mL/min.

### MALDI-MS Target Plate Engineering to Incorporate the BLI Chip

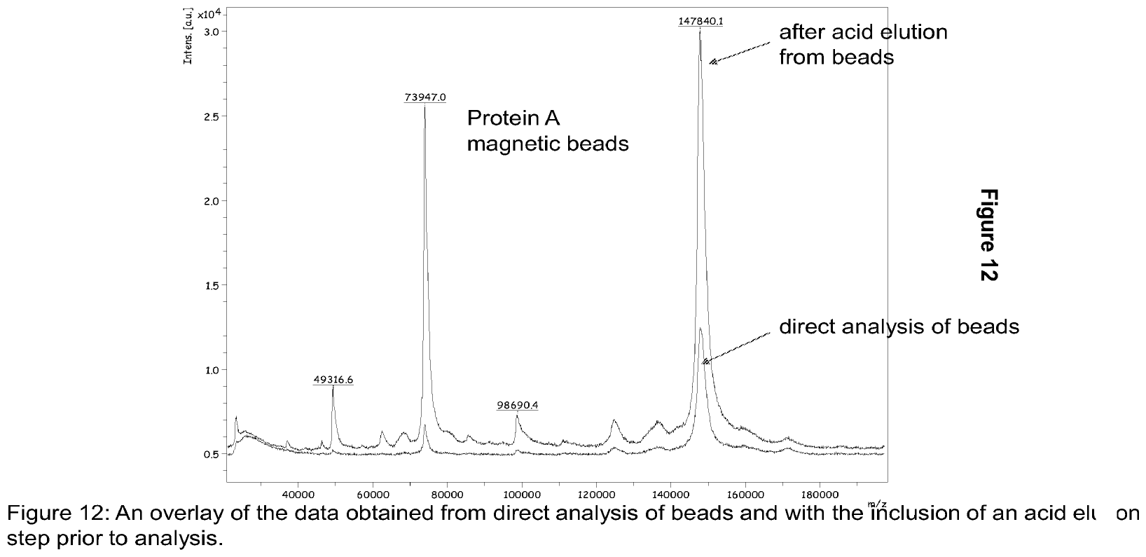

Two options for mounting a BLI chip to a MALDI plate were developed: First, the BLI chip was surface mounted into a pocket milled into the front of a MALDI plate. The depth of the pocket was such that the surface of the BLI chip was coincident with the front surface of the MALDI chip. The BLI chip was secured in the pocket by two M2 screws that pass through pre-existing holes in the BLI chip and screw into tapped holes in the MALDI plate (FIG. 12).

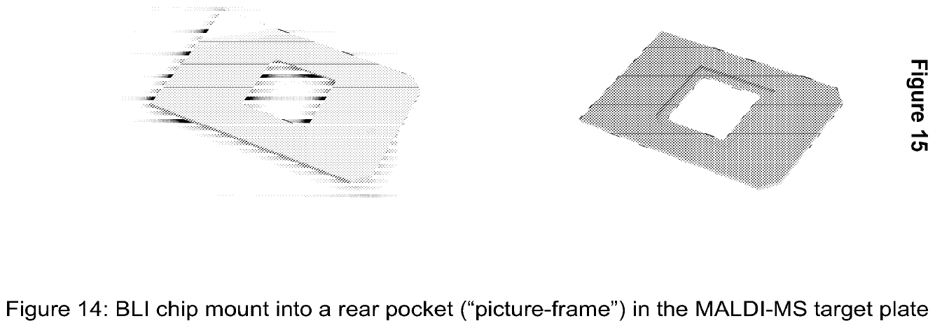

Second, to Mount the BLI chip into a pocket in the rear of the MALDI plate. The periphery of the pocket was not milled all the way through the MALDI plate, such that it captured the BLI chip in a “picture frame.” The center of the pocket was milled all the way through the MALDI plate so that the front of the BLI chip was exposed, with the surface of the microfluidic components being coincident with the surface of the MALDI plate (FIG. 14). The BLI chip was secured in the pocket when the MALDI plate was mounted to the MALDI plate carrier.

### Example 1

### Evaluation of MALDI-MS for Clone Evaluation

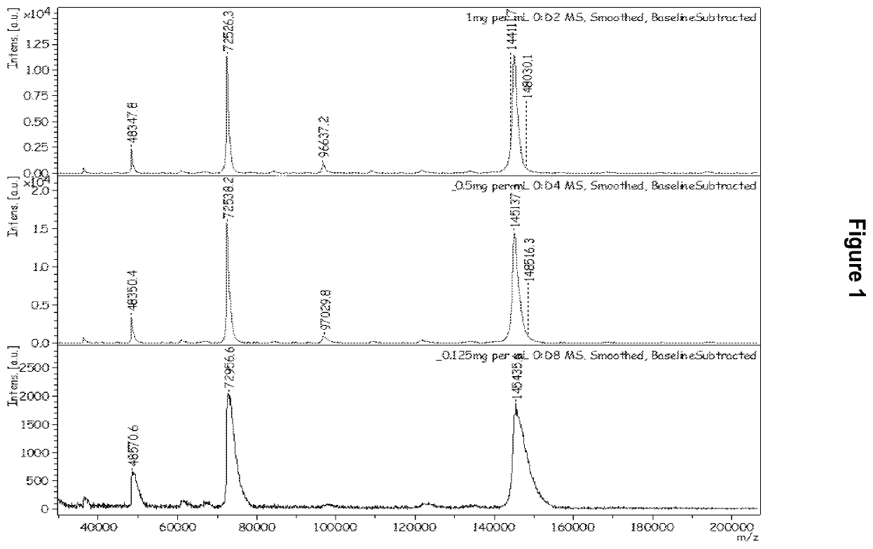

Experiments were designed to determine whether MALDI-MS in conjunction with a fluidic device could be used for clonal evaluation. First, a mAb was diluted to 1 mg/ml in 1×PBS, and then serial diluted into 0.5 mg/mL, 0.25 mg/mL and 0.125 mg/mL by mixing 5 μL of the protein with 5 μL of 1×PBS. MALDI was carried out as described above. For MALDI plate spotting, 1 μL of each protein concentration was mixed with 1 μL of sinapinic acid (10 mg/mL in 50% MeCN in 0.1% TFA) before 1 μL was spotted onto the MALDI plate, air dried and Mw measured. This is equivalent to spotting 0.5 μg, 0.25 μg, 0.125 μg and 0.0625 μg of mAb onto the well. The data are shown in the mass spectra in FIG. 1. The 0.25 mg/ml material did not give a “good spot”, and the lower panel has broader peaks. This experiment shows that mAb were observed on the MALDI-MS plate and this method can be used in conjunction with a fluidic device to detect the mAb.

### Example 2

### Evaluation of MALDI-MS for Directly Monitoring mAb Expression in Conditioned Media

The conditioned media from cells expressing a monoclonal antibody (denoted herein as mAb 2) was analyzed using MALDI-MS as described above. The conditioned media was collected after 11 days in culture and directly spotted onto the MALDI plate, matrix added, sample air dried and Mw measured.

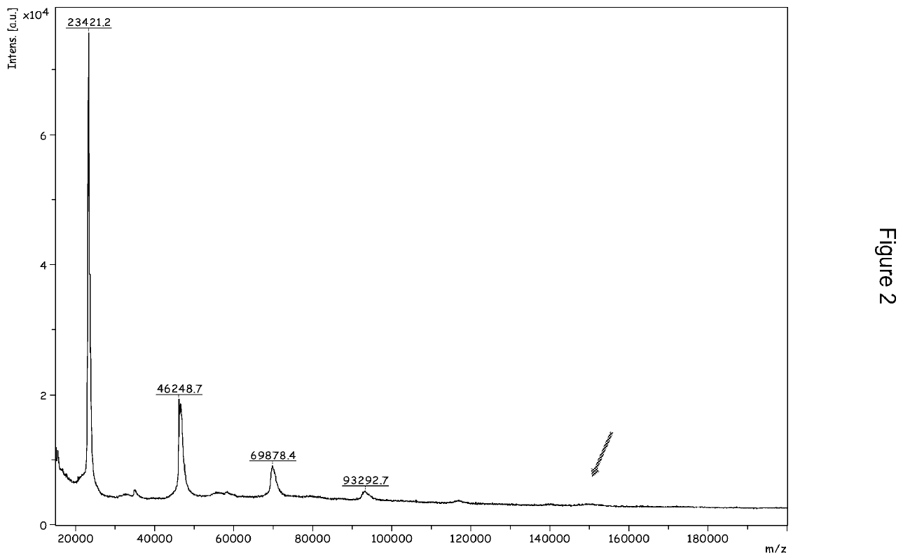

As shown in FIG. 1, the mass spectra of the non-reduced sample shows that the mass spectra was dominated by the light chain signal while the signal of the intact mAb was suppressed (indicated by the arrow). To determine what was interfering with the intact mAb signal, the Day−11 conditioned media was analyzed using RP-HPLC and LC-ESI-MS. RP-HPLC analysis of the conditioned media showed its complexity with materials identified to be free cysteinylated light chain, free glutathionylated light chain, light chain dimer and Ab. FIG. 2A shows a RP-HPLC chromatogram where the conditioned media separated into three major peaks (peak 1, 2, 3a and 3b). FIG. 2B shows a reducing SDS-PAGE analysis of the RP-HPLC fractions demonstrating that the expressed mAb eluted in peak 3.

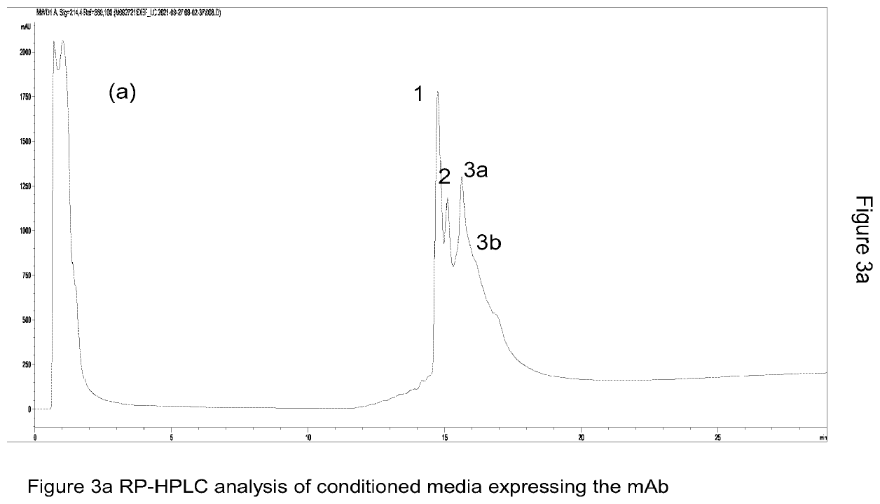

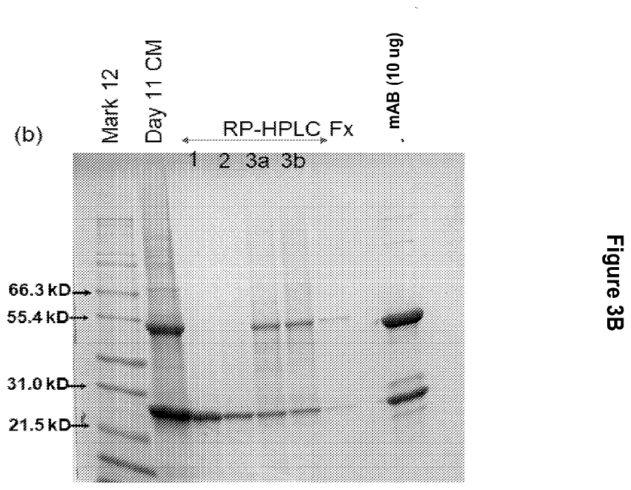

FIG. 3A & B provide LC-ESI-MS data identifying the RP-HPLC fractions from peaks 1 and 2. FIG. 3A shows that peak 1 contained a mixture of cysteinylated and glutathionylated light chain. FIG. 3B shows that peak 2 corresponded to the light chain dimer and the dimer with either a cysteine or glutathione adduct. Therefore, analysis of the conditioned media showed it to be a complex mixture containing an abundance of overly expressed light chain (free cysteinylated light chain, free glutathionylated light chain, light chain dimer), mAb and a polymer like material. Co-elution of the polymer like material with the mAb in peak 3 of the RP-HPLC fractions probably explains why Mw measurement of the mAb was unsuccessful. Therefore, in order to analyze mAb expression in the conditioned media by MALDI-MS, a preliminary purification step is beneficial.

### Example 3

### MALDI-MS Analysis of mAb Following a Partial Purification Step

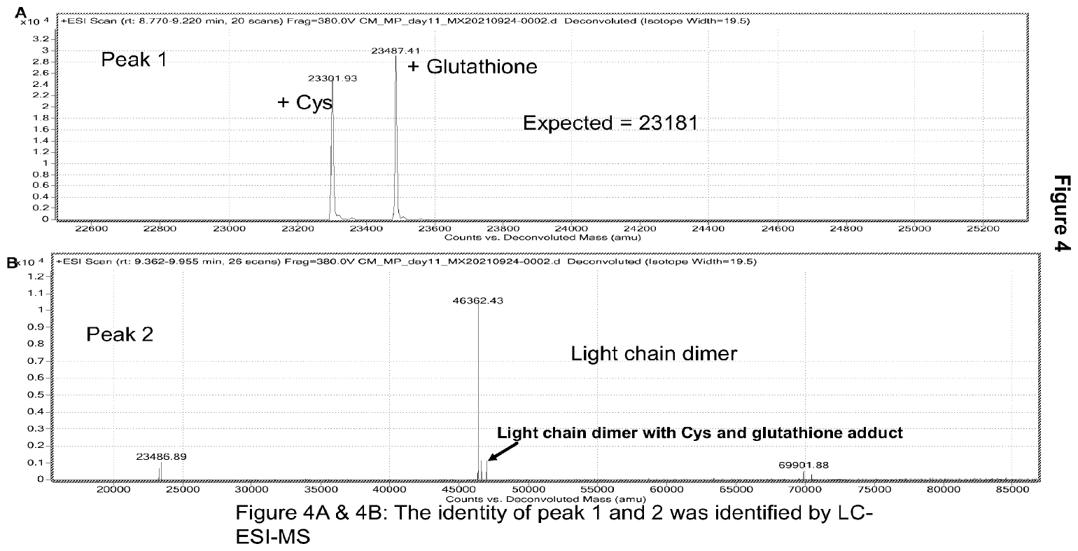

To improve sensitivity of the MALDI-MS analysis, the protein was partially purified prior to subjecting the sample to MALDI-MS. As described above, the 11 day conditioned media was contacted with Protein A/G spin column (Pierce). FIG. 4 demonstrates that the expressed mAb were recovered from the conditioned media using a protein A/G spin column. The chromatogram in the upper panel shows that the light chain derivatives were recovered in the unbound fraction and the bound mAb was detected in the 1% acetic acid eluate whose Mw is shown in the ESI spectrum in the bottom panel. Therefore, Protein A/G magnetic beads were used for a preliminary purification step of the mAb that would be amenable to subsequent MALDI-MS characterization.

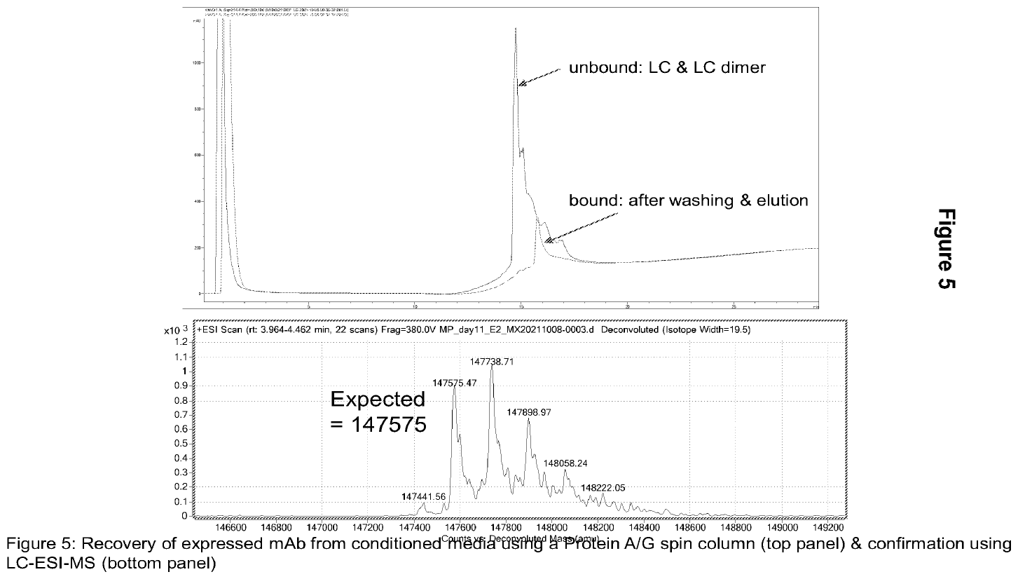

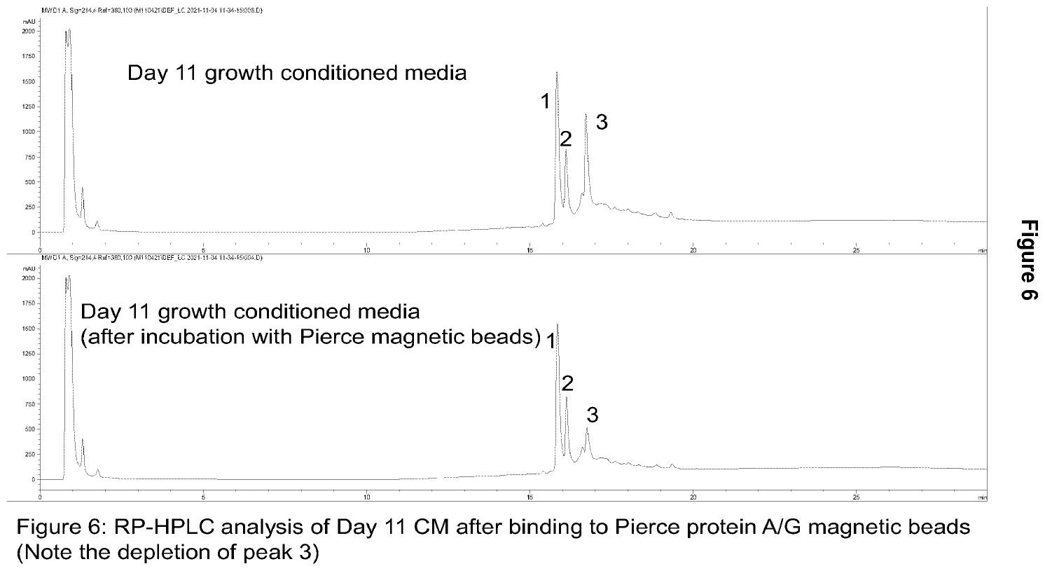

The beads were added to the conditioned media to capture the mAb and then collected using a magnetic stand, washed, mixed with 20 μL PBS and 1 μL of the slurry spotted onto a MALDI plate. This was followed by the addition of 2 μL Sinapinic acid MALDI matrix and 1 μL, 5% acetic acid to elute the bound mAb prior to analysis by RP-HPLC and LC-ESI-MS. The spots were then air-dried and subjected to MALDI-MS. FIG. 5 is a RP-HPLC chromatogram showing that Protein A/G magnetic beads recovered up to 80% of the mAb from the conditioned media. The red chromatogram in the bottom panel is RP-HPLC analysis. FIG. 6 shows a mass spectra of mAb analyzed directly with MALDI-MS while bound to Protein A/G magnetic beads.

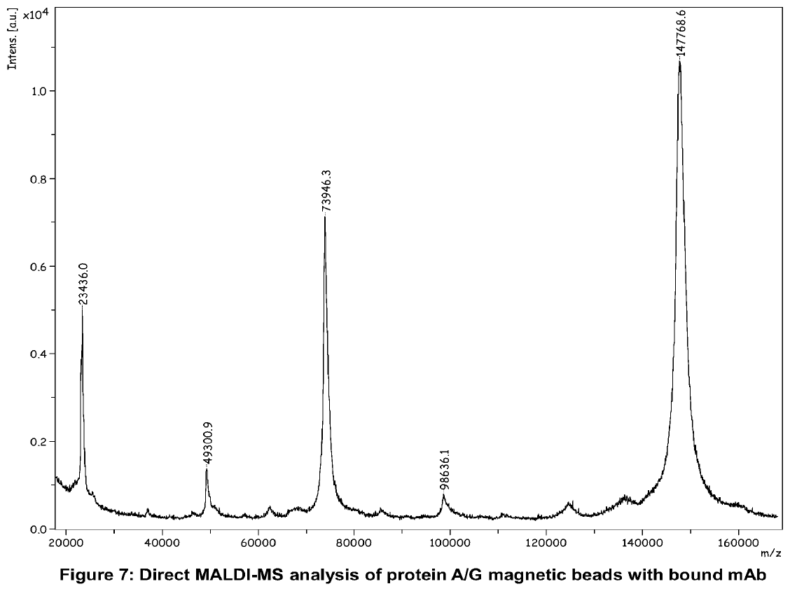

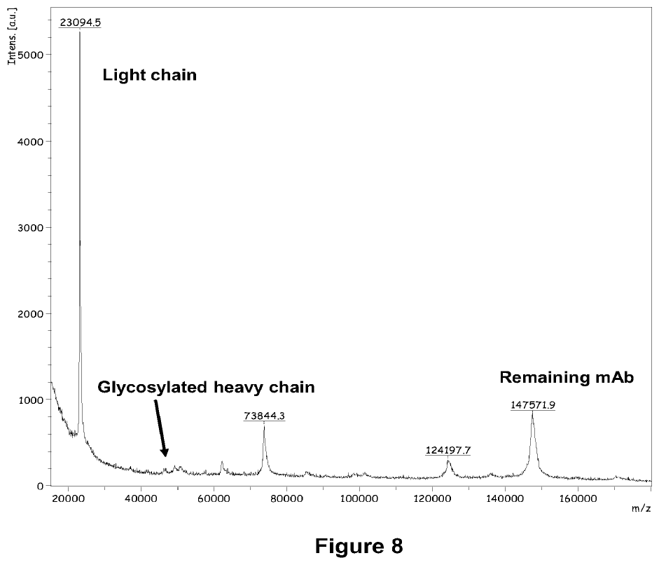

To test whether the mAb can be detected after direct reduction on the beads, 20 mM TCEP was added to matrix that was added to beads spotted on a MALDI plate. Reduction was allowed to occur at room temperature and the spot air-dried before being subjected to MALDI-MS. FIG. 7 shows detection of the light chain and a suppressed signal from the heavy chain, possibly due to glycosylation and therefore reduction in ion signal and a peak from the intact mAb due to incomplete reduction of the mAb which can be further optimized. In FIG. 7, the 49300 ion is the 3+ ion of the mAb. The 98636 peak is the (Fab)₂. The 23436 ion is the light chain. Their relative amount is not indicative of their relative proportion. These data demonstrate that the constituent components of the bead bound mAb can be readily analyzed directly after on-plate reduction.

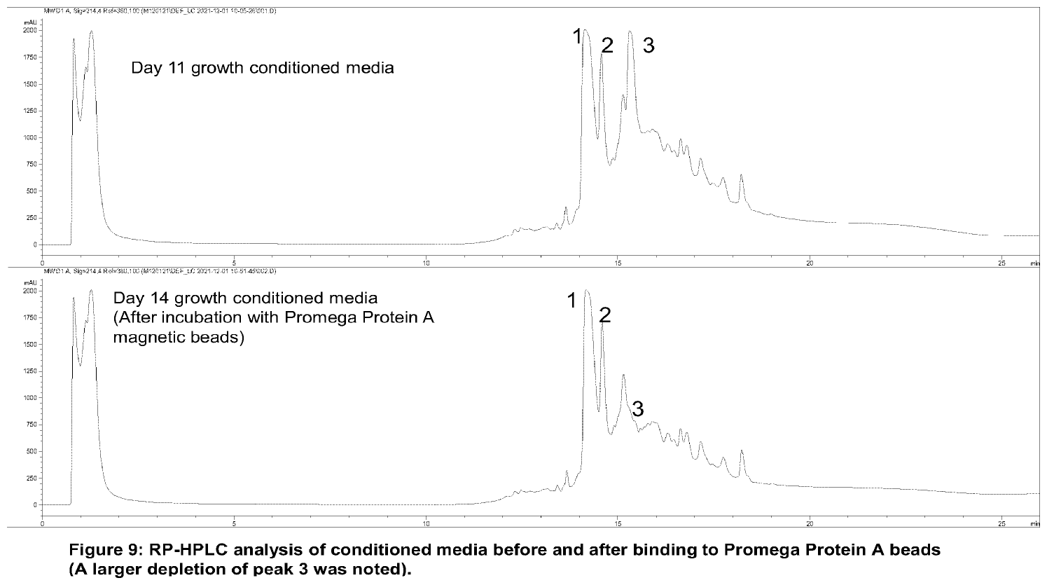

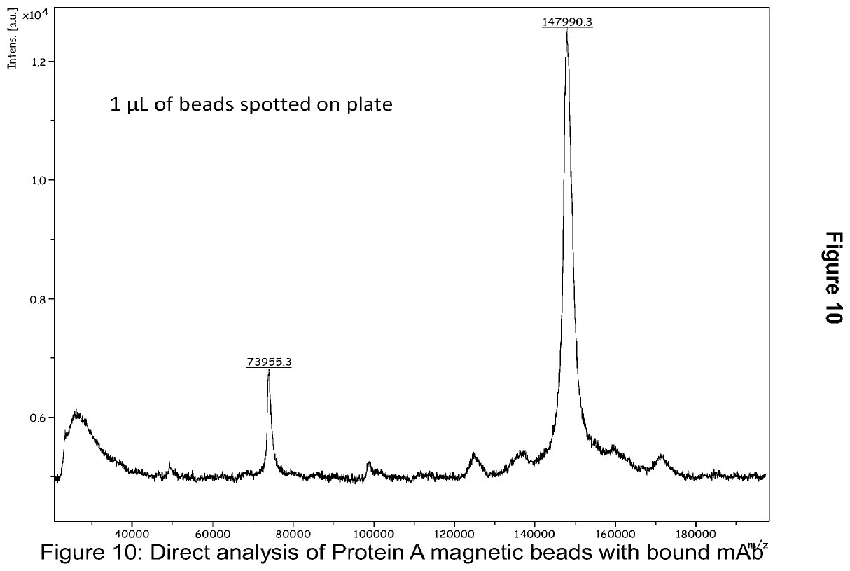

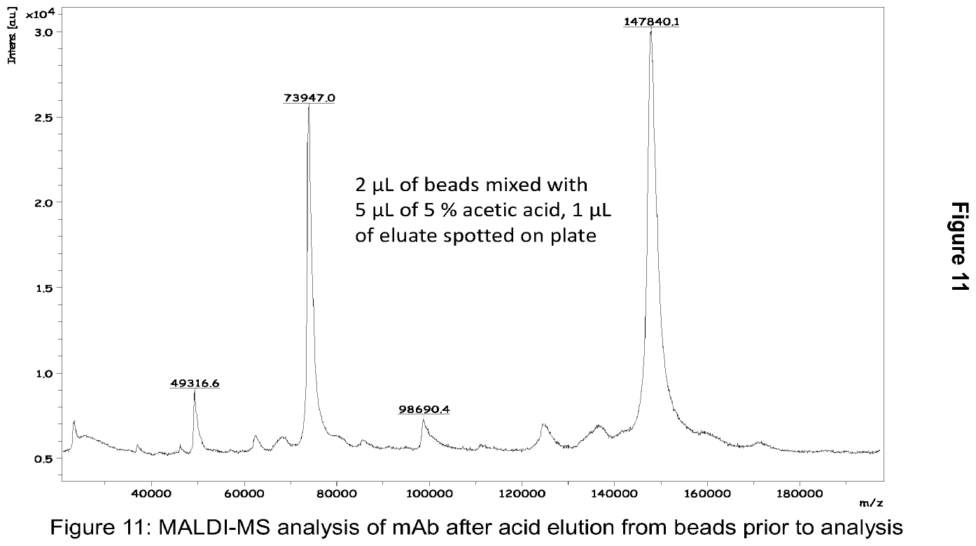

The aforementioned bead experiments were repeated using Promega Protein A beads under the same experimental conditions. Here too, depletion of peak 3 shows that most of the mAb in the conditioned media was bound to the beads (FIG. 8). Direct analysis of the beads demonstrated similar results as described using the Protein A/G beads above (see FIG. 9). In a different experiment, 2 μL of the beads with bound mAb was transferred to a microfuge tube and 5 μL, 5% acetic acid acetic acid added. After the beads settled, 1 μL of the solution was subjected to MALDI-MS which yielded 3-fold more intense signal, (see FIGS. 10 and 11). Therefore, partial purification of mAb from conditioned media with Protein A magnetic beads can be readily employed prior to their analysis by MALDI-MS.

### Example 4

### MALDI-MS Analysis of mAb Following Reduction on the Glass Cover of the MALDI-MS Plate

An assay was carried out to determine if the mAb can be analyzed by MALDI-MS after reduction of the mAb directly on a glass cover on a MALDI-MS plate. The mAB was diluted in 10 μL of 1% acetic acid and 0.1 M TCEP in 50% acetonitril, and 1 μL of the slurry spotted onto a MALDI plate. Subsequently, sinapinic acid (10 mg per mL in 0.1% TFA 50% acetonitrile) was directly layered onto the spot of mAB on the glass cover and allowed to air dry prior to being subjected to MALDI-MS.

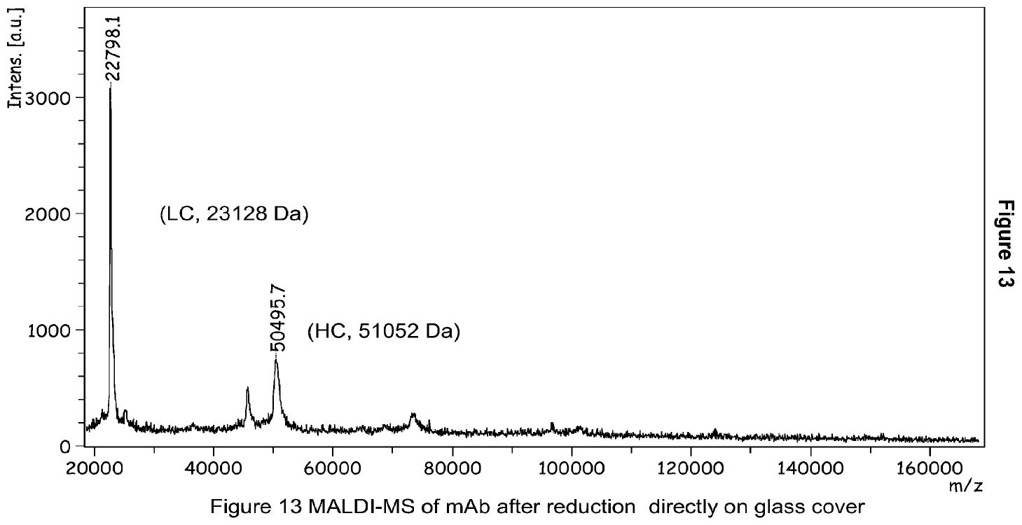

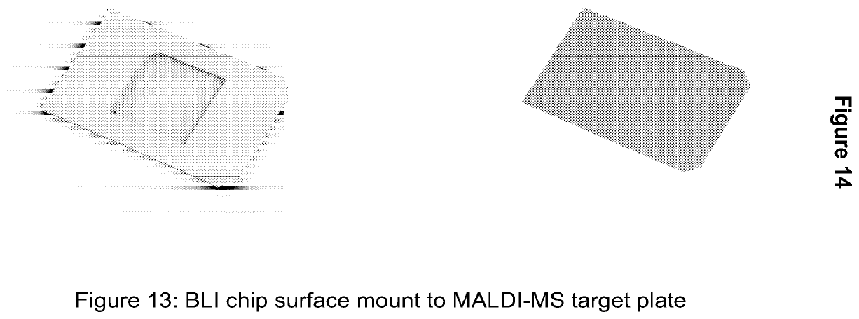

FIG. 13 shows detection of the light chain and a suppressed signal from the heavy chain, possibly due to glycosylation and therefore reduction in ion signal and a peak from the intact mAb due to incomplete reduction of the mAb which can be further optimized. The 22798.1 peak is the light chain and the 50495.7 peak is the heavy chain. Their relative amount is not indicative of their relative proportion. These data demonstrate that the constituent components of the mAb can be readily analyzed directly after on-plate reduction.

### Example 5

### MALDI-MS Screening of Antibodies/Plasma Cells in a Microfluidic Device.

The proof-of-concept data in the preceding examples showing that MALDI-MS can be used to accurately analyze the Mw of mAb in conditioned media and directly on Protein A/G beads in reducing and non-reducing condition indicate that a MALDI-MS assay can be adapted for scoring secreted antibody on a BLI chip mounted on a MALDI target plate.

Fitting a BLI Chip into a MALDI target plate: The spatial focus of the MALDI laser is 10-20 μm in diameter while the BLI pens are approximately 60 μm in width, therefore the laser dimensions “fit” inside the BLI pens. Antibody producing cells are imported into a microfluidic device by flowing the suspension into an inlet and stopping the flow when the cells are located within the flow region/microfluidic channels. Cells are then loaded into the sequestration pens, with a target of one cell per pen. The cells are moved from the flow region/microfluidic channels into the isolation regions of the sequestration pens using light-activated DEP force (OEP technology).

Assaying the cells using MALDI-MS: A few days following penning, the cells are assayed to analyze the molecular weight of secreted antibodies which are then compared against the expected molecular weight to determine whether the right antibody is being expressed. Further, MALDI-MS analysis allows for the detection of chain mispairings as well as identification of post translational modifications. On plate/chip reduction of the antibody improves the mass accuracy and sensitivity.

Moving the Fc-capture Beads and/or ProA/G capture beads out of the BLI pens and on to a separate MALDI-MS target plate or in to a 96 and/or 384 well plate for (reversed-phase liquid chromatographic mass spectrometry (11, 12) rpLC-MS analyses. Moving the bead captured mAbs out of the BLI chip/pens allows for “pooling” and therefore greater MS sensitivity. Optionally, the beads may be barcoded so that clone of origin may be identified for protein on each bead. Mobile phases for rpLC-MS are acidic in nature, therefore this is compatible with eluting the captured mAb from the anti-Fc and/or ProA/G bead.

### REFERENCES

1. Jorgolli, M.; Nevill, T.; Winters, A.; Chen, I.; Chong, S.; Lin, F. F.; Mock, M.; Chen, C.; Le, K.; Tan, C.; Jess, P.; Xu, H.; Hamburger, A.; Stevens, J.; Munro, T.; Wu, M.; Tagari, P.; Miranda, L. P., Nanoscale integration of single cell biologics discovery processes using optofluidic manipulation and monitoring. Biotechnol Bioeng 2019, 116 (9), 2393-2411.
2. Cohen, L. H.; Gusev, A. I., Small molecule analysis by MALDI mass spectrometry. Anal Bioanal Chem 2002, 373 (7), 571-86.
3. Cohen, S. L.; Chait, B. T., Influence of matrix solution conditions on the MALDI-MS analysis of peptides and proteins. Anal Chem 1996, 68 (1), 31-7.
4. Karas, M.; Bachmann, D.; Hillenkamp, F., Influence of the Wavelength in High-Irradiance Ultraviolet Laser Desorption Mass Spectrometry of Organic Molecules. Analytcal Chemistry 1985, 57, 2935-2839.
5. Buck, A.; Ly, A.; Balluff, B.; Sun, N.; Gorzolka, K.; Feuchtinger, A.; Janssen, K. P.; Kuppen, P. J.; van de Velde, C. J.; Weirich, G.; Erlmeier, F.; Langer, R.; Aubele, M.; Zitzelsberger, H.; Aichler, M.; Walch, A., High-resolution MALDI-FT-ICR MS imaging for the analysis of metabolites from formalin-fixed, paraffin-embedded clinical tissue samples. J Pathol 2015, 237 (1), 123-32.
6. Korte, A. R.; Yandeau-Nelson, M. D.; Nikolau, B. J.; Lee, Y. J., Subcellular-level resolution MALDI-MS imaging of maize leaf metabolites by MALDI-linear ion trap-Orbitrap mass spectrometer. Anal Bioanal Chem 2015, 407 (8), 2301-9.
7. Aichler, M.; Walch, A., MALDI Imaging mass spectrometry: current frontiers and perspectives in pathology research and practice. Lab Invest 2015, 95 (4), 422-31.
8. Guenther, S.; Koestler, M.; Schulz, O.; Spengler, B., Laser spot size and laser power dependence of ion formation in high resolution MALDI imaging. Internation Journal of Mass Spectrometry 2010, 294 (1), 7-15.
9. Zavalin, A.; Yang, J.; Hayden, K.; Vestal, M.; Caprioli, R. M., Tissue protein imaging at 1 mum laser spot diameter for high spatial resolution and high imaging speed using transmission geometry MALDI TOF MS. Anal Bioanal Chem 2015, 407 (8), 2337-42.
10. Prentice, B. M.; Chumbley, C. W.; Caprioli, R. M., High-speed MALDI MS/MS imaging mass spectrometry using continuous raster sampling. J Mass Spectrom 2015, 50 (4), 703-10.
11. Dillon, T. M.; Bondarenko, P. V.; Rehder, D. S.; Pipes, G. D.; Kleemann, G. R.; Ricci, M. S., Optimization of a reversed-phase high-performance liquid chromatography/mass spectrometry method for characterizing recombinant antibody heterogeneity and stability. J Chromatogr A 2006, 1120 (1-2), 112-20.
12. Campuzano, I. D. G.; Sandoval, W., Denaturing and Native Mass Spectrometric Analytics for Biotherapeutic Drug Discovery Research: Historical, Current, and Future Personal Perspectives. J Am Soc Mass Spectrom 2021, 32 (8), 1861-1885.

What is claimed:

1. A method of detecting a protein in a sample, the method comprising subjecting the sample to mass spectrometry while the sample is in a fluidic device.

2. A method of detecting protein in samples in a fluidic device comprising:
contacting samples in the device with solid supports, each solid support comprising a ligand for the protein, whereby the ligand binds to the protein of the sample, and wherein each solid support comprising the ligand bound to the protein comprises a unique barcode different from other solid supports comprising the ligand bound to the protein;
transporting the solid support comprising the ligand bound to the protein from a first location in the fluidic device to a second location, wherein the second location is in the fluidic device or outside of the fluidic device; and
subjecting the sample to mass spectrometry at the second location.

3. The method of claim 1 or 2 wherein the protein in the sample is reduced.

4. The method of claim 1 or 2, wherein the method further comprises reducing the protein in the sample prior to subjecting the sample to mass spectrometry.

5. The method of claim 3 or 4 wherein the protein is reduced in situ or the protein is reduced in the mass spectrometry device.

6. The method of any of the preceding claims, wherein the mass spectrometry is matrix-assisted laser desorption mass spectrometry/ionization time-of-flight/time-of-flight (MALDI-TOF/TOF).

7. The method of any one of claims 1-6, wherein the mass spectrometry is liquid chromatography-mass spectrometry/mass-spectrometry (rpLC-MS/MS), hydrophobic interaction chromatography-mass spectrometry (HIC-MS) or cation exchange chromatography-mass spectrometry (CEX-MS)

8. The method of claim 7, wherein the LC-MS/MS comprises electrospray ionization.

9. The method of any of the preceding claims, wherein the sample comprises conditioned media.

10. The method of any of the preceding claims, wherein the protein comprises or consists of a large peptide, antibody, antibody fragment, antibody fusion peptide or antigen-binding fragment thereof.

11. The method of claim 10, wherein the antibody is a polyclonal or monoclonal antibody.

12. The method of any one of claims 1-11, wherein the protein is partially purified prior to being subjected to mass spectrometry.

13. The method of any one of claims 1-11, wherein the method further comprises partially purifying the protein.

14. The method of claim 13, wherein the partial purification comprises contacting the sample with a solid support comprising a ligand for the protein, prior to subjecting the sample to mass spectrometry.

15. The method of any one of claim 14, wherein the solid support comprising the ligand bound to the protein comprises a unique barcode, wherein the partial purification further comprises other solid supports comprising ligand bound to other protein and comprising other barcodes that are different from the unique barcode.

16. The method of claim 15, wherein subjecting the sample to mass spectrometry comprises subjecting a batch comprising the solid support, wherein the solid support comprising the ligand bound to the protein comprises a unique barcode and wherein the solid support further comprises at least some of the other solid supports comprising ligand bound to other protein and comprising other barcodes.

17. The method of any one of claims 14-16, the partial purification further comprising transporting the solid support comprising the ligand bound to the protein from a first location in the fluidic device to a second location in the fluidic device prior to subjecting the sample to mass spectrometry.

18. The method of any one of claims 14-16, the partial purification further comprising transporting the solid support comprising the ligand bound to the protein from a first location in the fluidic device to a second location on a multi-well plate prior to subjecting the sample to mass spectrometry.

19. The method of any one of claims 14-18 wherein the solid support comprises anti-FC protein, protein A or protein G, or wherein the solid support comprises protein A or protein G.

20. The method of claim 2 wherein the solid support comprises anti-FC protein, protein A or protein G, or wherein the solid support comprises protein A or protein G.

21. The method of any of the preceding claims, wherein the fluidic device is a microfluidic chip or sequestration pen.

22. The method of any one of the preceding claims, wherein the fluidic device comprises a silicon surface, and wherein the sample is disposed on the silicon surface.

23. A mass spectrometry plate comprising a fluidic device mounted to the surface of the mass spectrometry plate.

24. The mass spectrometry plate of claim 23, wherein the fluidic device is open, thereby configuring an interior of the fluidic device to be directly engaged by a mass spectrometer.

25. The mass spectrometry plate of claim 23 or 24, wherein the mass spectrometry is matrix-assisted laser desorption/ionization time-of-flight/time-of-flight (MALDI-TOF/TOF).

26. The mass spectrometry plate of claim 23 or 24, wherein the mass spectrometry is liquid chromatography-mass spectrometry/mass-spectrometry (rpLC-MS/MS), hydrophobic interaction chromatography-mass spectrometry (HIC-MS) or cation exchange chromatography-mass spectrometry (CEX-MS).

27. The mass spectrometry plate of claim 26, wherein the LC-MS/MS comprises electrospray ionization.

28. A system for detecting protein in a sample wherein the system comprises a mass spectrometry plate of any one of claims 23-27.

29. The system of claim 28 further comprising a control or a protein standard.

30. A kit comprising a mass spectrometry plate of any one of claims 23-29.

31. The system or kit of any one of claims 28-30, further comprising a solid support comprising a ligand, such as anti-FC protein, protein A or protein G.

32. The method of any one of claims 2, 14-20, or system or kit of claim 31, wherein the solid support comprises a bead, such as a bead having a mean diameter of at least about 1 μm.
